(19) United States
(12) Patent Application Publication
(10) Pub. No.: US 2009/0171336 A1
(43) Pub. Date: Jul. 2, 2009

(54) CATHETERS AND MANUFACTURING THEREOF

(75) Inventor: Jan Weber, Maastricht (NL)

Correspondence Address: CROMPTON, SEAGER & TUFTE, LLC, 1221 NICOLLET AVENUE, SUITE 800, MINNEAPOLIS, MN, 55403-2420, US

(73) Assignee: Boston Scientific Scimed, Inc., Maple Grove, MN (US)

(21) Appl. No.: 11/964,308

(22) Filed: Dec. 26, 2007

(51) Int. Cl. A61B 18/08

(52) U.S. Cl. 606/27

(57) ABSTRACT

A catheter includes a longitudinally extending body having proximal and distal ends and defining at least one lumen that extends longitudinally from the proximal end through the body to the distal end and looping back to the proximal end. A liquid metal, e.g. an alloy of gallium and indium, such as galistan, is disposed in the lumen. In another aspect, a catheter includes a longitudinally extending body defining first and second lumens. An electrically driven device is coupled to a distal end of the body and is in electrical communication with the first and second lumens. A power source is in electrical communication with the first and second lumens and a liquid metal is disposed in the first and second lumens to provide an electrical conduit between the power source and electrically driven device. Each lumen may loop from a proximal end of the body to the distal end back to the proximal end.

### TECHNICAL FIELD

This disclosure relates to catheters and manufacturing of catheters.

### BACKGROUND

A catheter is a tube that can be inserted into a body cavity, duct or vessel to allow drainage or injection of fluids or access by surgical instruments. Catheterization may be used for draining urine from a urinary bladder, draining fluid collections (e.g. an abdominal abscess), administering intravenous fluids or medication, direct measurement of blood pressure or intracranial pressure, angioplasty, angiography, balloon septostomy, and balloon sinuplasty, inter alia, for example. A balloon catheter is a type of catheter with an inflatable “balloon” at its tip which is used during a catheterization procedure to enlarge a narrow opening or passage within the body.

### SUMMARY

In one aspect, a catheter includes a longitudinally extending body having proximal and distal ends and defining at least one lumen. The lumen extends longitudinally from the proximal end through the body to the distal end and looping back to the proximal end. A liquid metal is disposed in the lumen.

Implementations of this aspect of the disclosure may include one or more of the following features. In some implementations, the liquid metal comprises an alloy of gallium and indium, e.g. galistan. In some examples, the catheter includes a power source in electrical communication with the liquid metal, which provides an electrical conduit for a current. The liquid metal may be flowed though the lumen, where a flow rate of the liquid metal controls a catheter temperature. The ability to flush the liquid metal in and out of the lumen may be useful, e.g., in MRI applications, where long solid metallic conductors may be locally heated by standing radio frequency (RF) waves in the system. A flowed liquid metal conductor tends to prevent localized heating by moving conducted heat away from a source of conduction. In some examples, the lumen has a relatively narrower defined cross-section in a distal portion of the body than in a proximal portion of the body. When the liquid metal is flowed though the lumen, the relatively narrower lumen in the distal portion of the body creates a flow resistance for the liquid metal, thereby allowing localized heating. When the catheter includes a power source in electrical communication with the liquid metal, the relatively narrower lumen in the distal portion of the body creates an electrical current resistance for the liquid metal electrical conduit, thereby allowing localized heating.

In some implementations, a wall thickness between the lumen and an exterior surface of the body is relatively thinner in a distal portion of the body than in a proximal portion of the body, thereby allowing greater thermal conduction between the exterior surface of the body and the liquid metal about the distal portion of the body than about the proximal portion of the body.

In some implementations, the catheter includes a balloon disposed at the distal end of the body. A wall of the balloon defines a fluid channel in fluid communication with the lumen. The balloon fluid channel may be in serial fluid communication with the lumen. In some instances, the balloon fluid channel has a relatively narrower defined cross-section than the lumen. When the catheter includes a power source in electrical communication with the liquid metal, the liquid metal provides an electrical conduit for current to heat tissue substantially about the balloon.

In another aspect, a catheter includes a longitudinally extending body having proximal and distal ends and defining first and second lumens extending longitudinally through the body. An electrically driven device (e.g. an actuator or sensor) is coupled to the distal end of the body and is in electrical communication with the first and second lumens. A power source is in electrical communication with the first and second lumens. A liquid metal is disposed in the first and second lumens and provides an electrical conduit between the power source and electrically driven device.

Implementations of this aspect of the disclosure may include one or more of the following features. In some implementations, the liquid metal comprises an alloy of gallium and indium, e.g. galistan. In some examples, each lumen extends longitudinally from the proximal end through the body to the distal end and loops back to the proximal end. The liquid metal is flowed through the first and second lumens, thereby moving conducted heat away from a source of thermal conduction. In some implementations, the catheter includes electrically insulative, thermally conductive particles disposed in the first and second lumens. The particles expand upon heating and obstruct the first and second lumens to disjoin the liquid metal, severing, e.g. temporarily or permanently, the electrical conduit between the power source and electrically driven device. In some examples, the particles chosen provide a reversible or a non-reversible system for severing the electrical conduit. For example, particles comprising polymer microcapsules filled with a blowing agent provide an irreversible system, and particles comprising paraffin or another type of wax provide a reversible system.

In yet another aspect, a catheter includes a longitudinally extending body having proximal and distal ends and defining first and second lumens. Each lumen extends longitudinally from the proximal end through the body to the distal end and loops back to the proximal end. An electrically driven device is coupled to the distal end of the body and is in electrical communication with the first and second lumens. A power source is in electrical communication with the first and second lumens. A liquid metal is flowed though the first and second lumens and provides an electrical conduit between the power source and electrically driven device. A flow rate of the liquid metal controls catheter temperature. In some implementations, the liquid metal comprises an alloy of gallium and indium, e.g. galistan.

In another aspect, an extruder head for an extruding device includes a head body defining at least one thermal conduction channel, a pump in fluid communication with the channel, and a liquid metal pumped through the channel to control an extruder head temperature. In some implementations, the liquid metal comprises an alloy of gallium and indium, e.g. galistan. This extruding device, or another extruding device, may include a cooling bath of liquid metal, e.g. an alloy of gallium and indium, such as galistan, for blow molding device an extrudate produced by the extruder head.

In another aspect, a blow molding device includes a manifold, at least one nozzle in fluid communication with the manifold, and a blow mold in fluid communication with the nozzle. The blow mold defines a blow mold cavity and at least one thermal conduction channel. A pump is in fluid communication with the channel and a liquid metal is pumped through the channel to control a blow molding device temperature. In some implementations, the liquid metal comprises an alloy of gallium and indium, e.g. galistan. The blow molding device may include a cooling bath of liquid metal, e.g. an alloy of gallium and indium, such as galistan, for cooling a product of the blow molding device.

In another aspect, a method of cooling an extruded polymer includes placing the extruded polymer into a bath of liquid metal, e.g. an alloy of gallium and indium, such as galistan, having a desired cooling temperature.

In another aspect, a method of heating an extruded polymer includes placing the extruded polymer into a bath of liquid metal, e.g. an alloy of gallium and indium, such as galistan, having a desired heating temperature.

The details of one or more implementations of the disclosure are set fourth in the accompanying drawings and the description below. Other features, objects, and advantages will be apparent from the description and drawings, and from the claims.

### DESCRIPTION OF DRAWINGS

FIG. 1 is a top view of a catheter.

FIG. 2 is a sectional view of a catheter.

FIGS. 3-7 are sectional views of catheters.

FIGS. 8-9 are sectional views of a lumen defined by a catheter.

FIG. 10 is a schematic view of an extruding device.

FIG. 11 is a sectional view of an extruder head.

FIG. 12 is a sectional view of a blow molding device.

FIG. 13 is a perspective view of a cooling/heating bath.

Like reference symbols in the various drawings indicate like elements.

### DETAILED DESCRIPTION

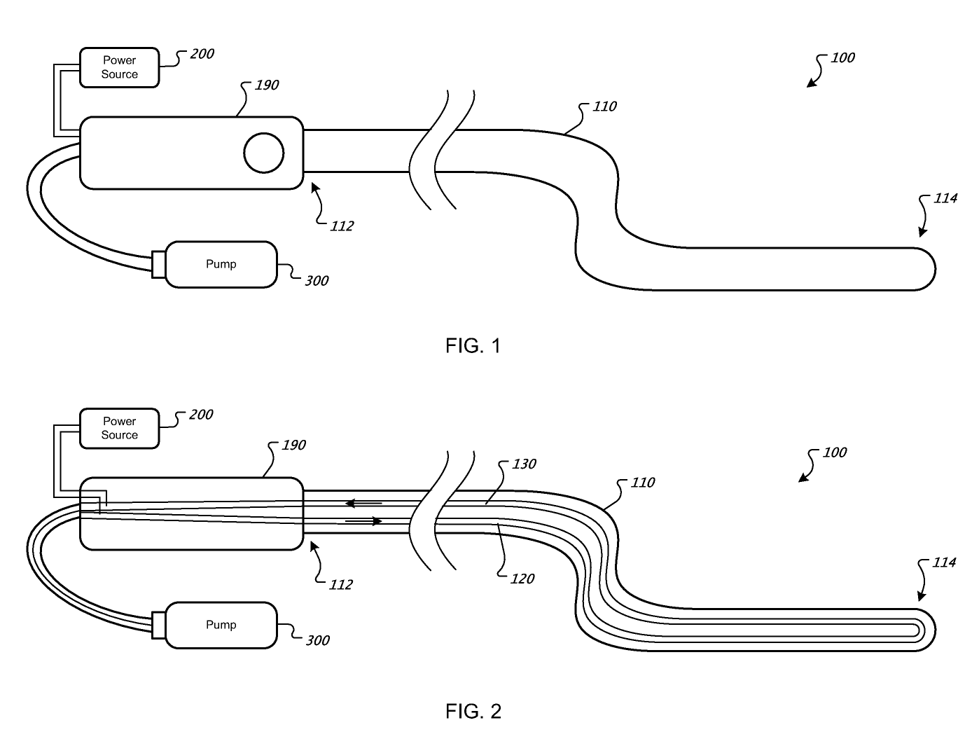

Referring to FIGS. 1-2, a catheter 100 includes a longitudinally extending body 110 having proximal and distal ends, 112 and 114 respectively, and defining at least one lumen 120. A handle 190 may be disposed at the proximal end 112 for holding and manipulating the catheter 100. The lumen 120 extends longitudinally from the proximal end 112 through the body 110 to the distal end 114 and loops back to the proximal end 112. A liquid metal 130 is disposed in the lumen 120. In some implementations, the liquid metal 130 comprises an alloy of gallium and indium, e.g. galistan, an eutectic alloy of gallium, indium, and tin. Galistan is a liquid at room temperature and will remain liquid between about −20° C. and 2000° C. Galistan does not contain mercury and is considered non-toxic. As a metallic substance, galistan conducts electricity and heat (having a thermal conductivity approximately 65 times greater than water). In other implementations, the liquid metal 130 comprises an alloy of gallium and indium. Preferably, the composition of the liquid metal 130 comprises between 65% to 75% by mass gallium and between 20% to 25% indium. Materials such as tin, copper, zinc and bismuth may also be present in relatively smaller percentages. One such preferred composition of the liquid metal 130 comprises 66% gallium, 20% indium, 11% tin, 1% copper, 1% zinc and 1% bismuth.

In some implementations, the catheter 100 includes a power source 200 in electrical communication with the liquid metal 130. The liquid metal 130 provides an electrical conduit or pathway though the catheter body 110 without contributing to the stiffness of the catheter 100. The catheter body 110 may be heated by delivering an electrical current through the liquid metal 130. In some instances, the liquid metal 130 is flowed though the lumen by a pump 300. A flow rate of the liquid metal 130 and/or a current level through the liquid metal 130 controls a catheter temperature.

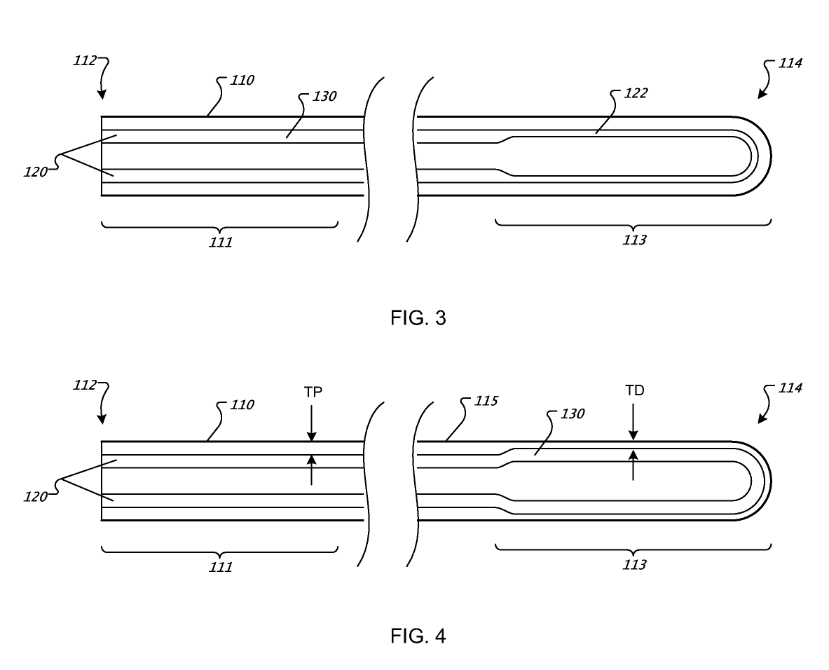

Referring to FIG. 3, the lumen 120 may have a relatively more narrow defined cross-section in a distal portion 113 of the body 110 than in a proximal portion 111 of the body 110. When the liquid metal 130 is flowed though the lumen 120, the relatively narrower lumen portion 122 in the distal portion 113 of the body 110 creates a flow resistance for the liquid metal 130, increasing thermal conduction in the distal portion 113 of the body 110. The flow rate of the liquid metal 130 may be controlled to obtain a desired temperature of the distal portion 113 of the body 110. When the catheter 100 includes a power source 300 in electrical communication with the liquid metal 130 and an electrical current is passed through the liquid metal 130, the relatively narrower lumen portion 122 creates an electrical resistance in the formed circuit. The temperature of the distal portion 113 of the body 110 may be controlled by adjusting the electrical current through the liquid metal 130.

Referring to FIG. 4, in some implementations, a wall thickness, TD, between the lumen 120 and an exterior surface 115 of the body is relatively thinner in the distal portion 113 of the body 110 than a wall thickness, TP, in the proximal portion 111 of the body 110, thereby allowing relatively greater thermal conduction between the exterior surface 115 of the body 110 and the liquid metal 130 about the distal portion 113 of the body 110 than about the proximal portion 111 of the body 110. When the liquid metal 130 is heated (e.g. via an electrical current or in a heated reservoir) the distal portion 113 of the body 110 may deliver heat to a localized portion of target tissue.

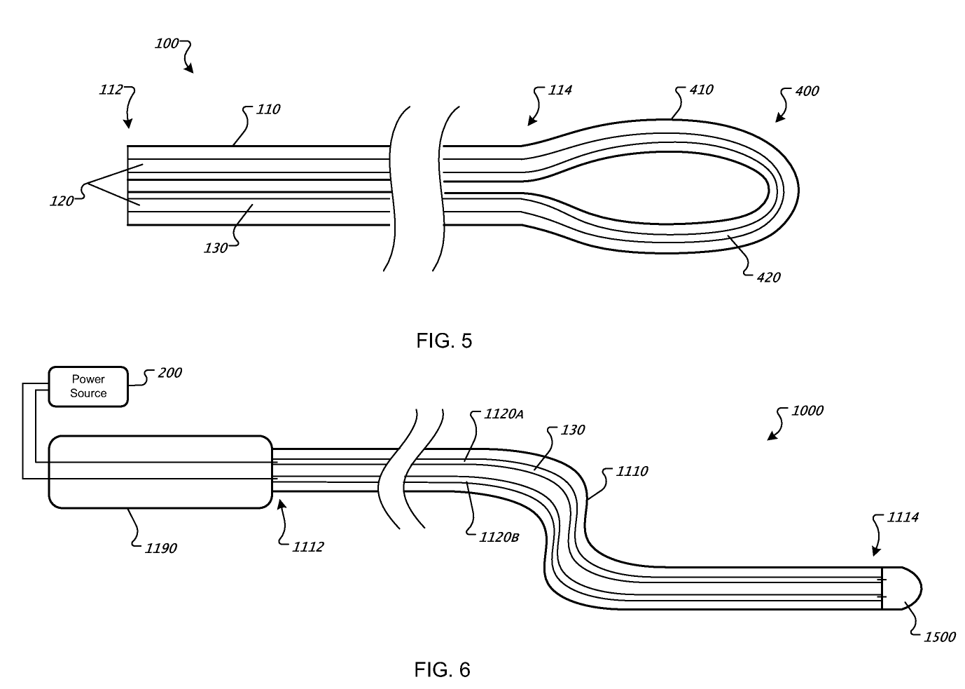

Referring to FIG. 5, in some implementations, the catheter 100 includes a balloon 400 disposed at the distal end 114 of the body 110. A wall 410 of the balloon 400 defines a fluid channel 420 in fluid communication with the lumen 120. In some examples, the balloon fluid channel 420 is in serial fluid communication with the lumen 120, as shown in FIG. 5. In other examples, the balloon fluid channel 420 is in parallel fluid communication with the lumen 120. In some implementations, the catheter 100 includes a power source 300 in electrical communication with the liquid metal 130. The liquid metal 130 provides an electrical conduit for current to heat tissue substantially about the balloon 400. The balloon fluid channel 420 may have a relatively narrower defined cross-section than the lumen 120 along all or some portions of the balloon fluid channel 420. When the liquid metal 130 is flowed though the lumen 120, the relatively narrower balloon fluid channel 420 creates a flow resistance for the liquid metal 130, increasing thermal conduction of the balloon 400. The flow rate of the liquid metal 130 may be controlled to obtain a desired temperature of the balloon 400. When the catheter 100 includes a power source 300 in electrical communication with the liquid metal 130 and a current is passed through the liquid metal 130, the relatively narrower balloon fluid channel 420 creates an electrical resistance in the formed circuit. The temperature of the balloon 400 may be controlled by adjusting the current through the liquid metal 130.

Referring to FIG. 6, in some implementations, a catheter 1000 includes a longitudinally extending body 1110 having proximal and distal ends, 1112 and 1114 respectively, and defining first and second lumens 1120A and 1120B, respectively, extending longitudinally through the body 1110. A handle 1190 may be disposed at the proximal end 112 for holding and manipulating the catheter 1000. An electrically driven device 1500 (e.g. an actuator or sensor) is coupled to the distal end 1114 of the body 1110 and is in electrical communication with the first and second lumens 1120A and 1120B, respectively. A power source 200 is in electrical communication with the first and second lumens 1120A and 1120B, respectively, and a liquid metal 130 disposed in the first and second lumens 1120A and 1120B, respectively. The liquid metal 130 provides an electrical conduit between the power source 200 and electrically driven device 1500. In some implementations, the liquid metal 130 comprises an alloy of gallium and indium, e.g. galistan.

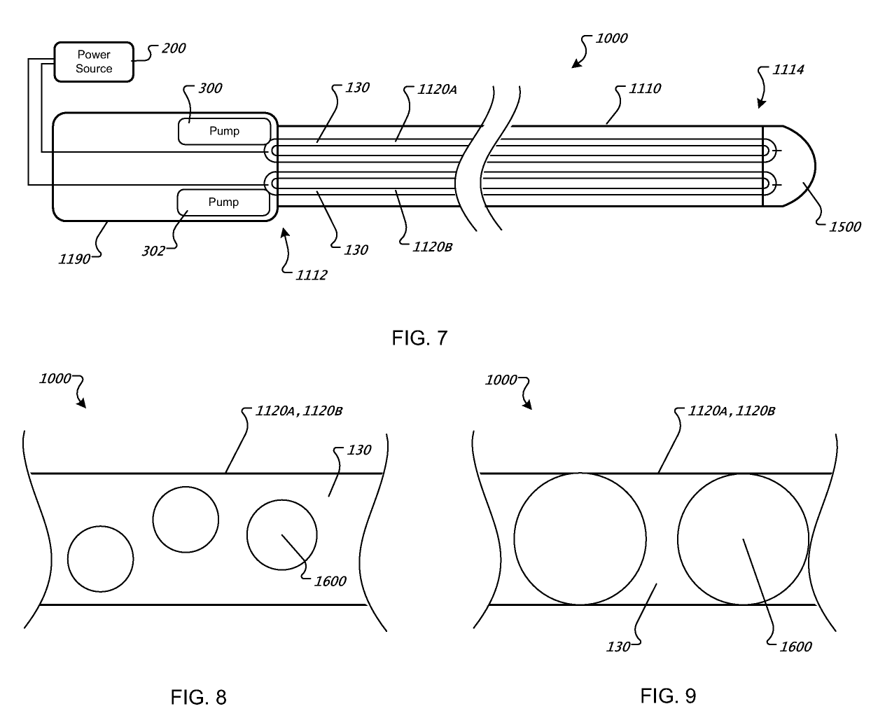

Referring to FIG. 7, in some implementations, the first and second lumens 1120A and 1120B, respectively, each extend longitudinally from the proximal end 1112 through the body 1110 to the distal end 1114 and loop back to the proximal end 1112. The liquid metal 130 is flowed through the first and second lumens 1120A and 1120B, respectively, (e.g. via pump 300 and 302 in fluid communication with the first and second lumens 1120A and 1120B, respectively) thereby carrying conducted heat away from a source of thermal conduction. A flow rate of the liquid metal 130 controls a catheter temperature.

Referring to FIGS. 8-9, in some implementations, the catheter 1000 includes electrically insulative, thermally conductive particles 1600 disposed in the first and second lumens 1120A and 1120B, respectively. The particles 1600 are formulated and/or constructed to expand upon heating to obstruct the first and second lumens 1120A and 1120B, respectively, thereby to disjoin the liquid metal 130, severing, temporarily or permanently, the electrical conduit between the power source 200 and electrically driven device 1500. In some examples, the particles 1600 chosen may provide a reversible or a non-reversible system. For example, particles 1600 comprising polymer microcapsules filled with a blowing agent, as described in U.S. patent application publication 2007/0154711 (having Ser. No. 10/595,910), the entire disclosure of which is incorporated herein by reference, will expand reversibly in the lumen 120 due to a rise in temperature of the liquid metal 130 (e.g. galistan), thereby blocking the lumen 120 permanently. In another example, demonstrating a reversible system, the particles 1600 of a suitable phase change material may be used to obtain sufficient thermal expansion and shrinkage to reversibly expand to restrict or block flow, and thereafter, with reduced temperature, to contract or shrink, to again permit flow. Paraffin is an example of a suitable material having a relatively large volume of expansion when going from solid to liquid with rise in temperature. Different formulations of paraffin with corresponding melting temperatures are disclosed in an article titled “Electrothermally Activated Paraffin Microactuators”, by Edwin T. Carlen and Carlos H. Mastrangelo, Journal of Microelectromechanical Systems, Vol. 11, No. 3, June 2002, the entire disclosure of which is incorporated herein by reference. Particles 1600 with a precise transition (swelling point) may be obtained by mixing different types of waxes. The molten paraffin may be enclosed in an elastic membrane. In some examples, a parylene membrane may be vapor deposited on wax (e.g. paraffin). The initial micro-sized spherical wax particles 1600 can be produced by rapidly cooling a molten wax-in-water solution stirred at high speed, after which a parylene or silicone layer is deposited on the particles as an outer membrane. The particles 1600 can then be sieved to obtain a desired dimensional particle size. In another fabrication method, was is vapor deposited on precisely templated particles 1600, e.g. silica microparticles, followed by vapor deposition of parylene (or another polymer) on the wax. In operation, once the wax becomes molten due to temperature rise of the liquid metal 130, the wax expands the parylene outer membrane.

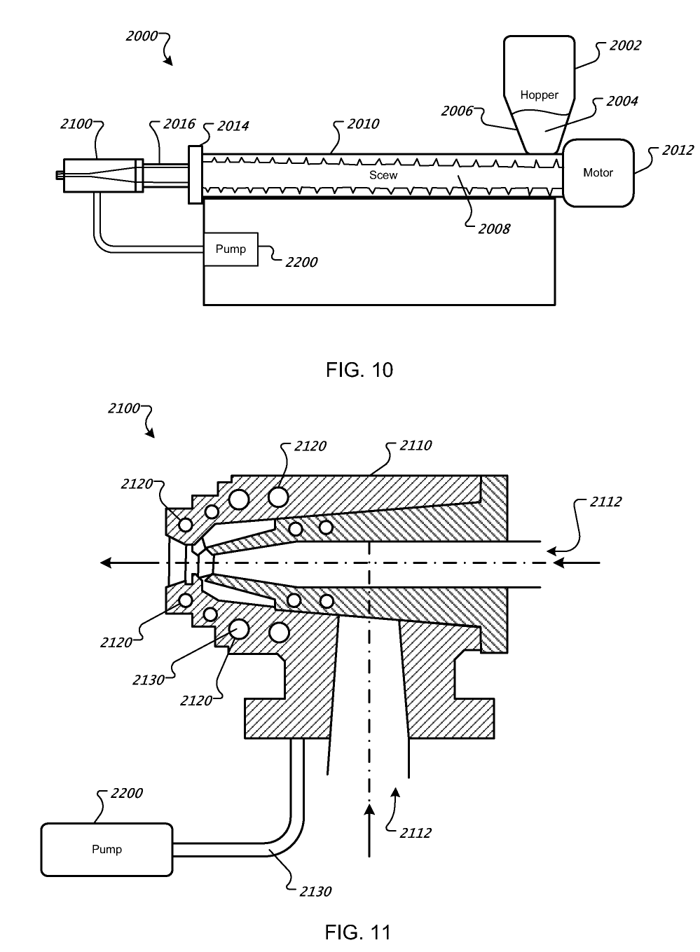

Referring to FIGS. 10-11, in some implementations, an extruder head 2100 for an extruding device 2000 includes a head body 2110 defining at least one thermal conduction channel 2120. The extruder head 2000 defines one or more extrusion channels 2112 configured to receive and form an extrusion substance 2004 (e.g. a polymer). A pump 2200 is in fluid communication with the channel 2120. A liquid metal 2130 is pumped through the channel 2120 to control an extruder head temperature (e.g. for heating or cooling the extrusion material). In some examples, the liquid metal 2130 comprises an alloy of gallium and indium, e.g. galistan. One example of an extruding device 2000 includes a hopper 2002 holding an extrusion material 2004 (e.g. plastic pellets), which moves through a feed throat 2006 and comes into contact with a screw 2008 housed by a screw housing 2010 and driven by a coupled motor 2012. The rotating screw 2008 forces the extrusion material 2004 forward in the screw housing 2010, which may be heated to a desired melt temperature of extrusion material 2004. The extrusion material 2004 melts gradually as it is pushed through the screw housing 2010 and passes through a breaker plate 2014 and a feed line 2016 to the extruder head 2100, which applies a profile for the final product.

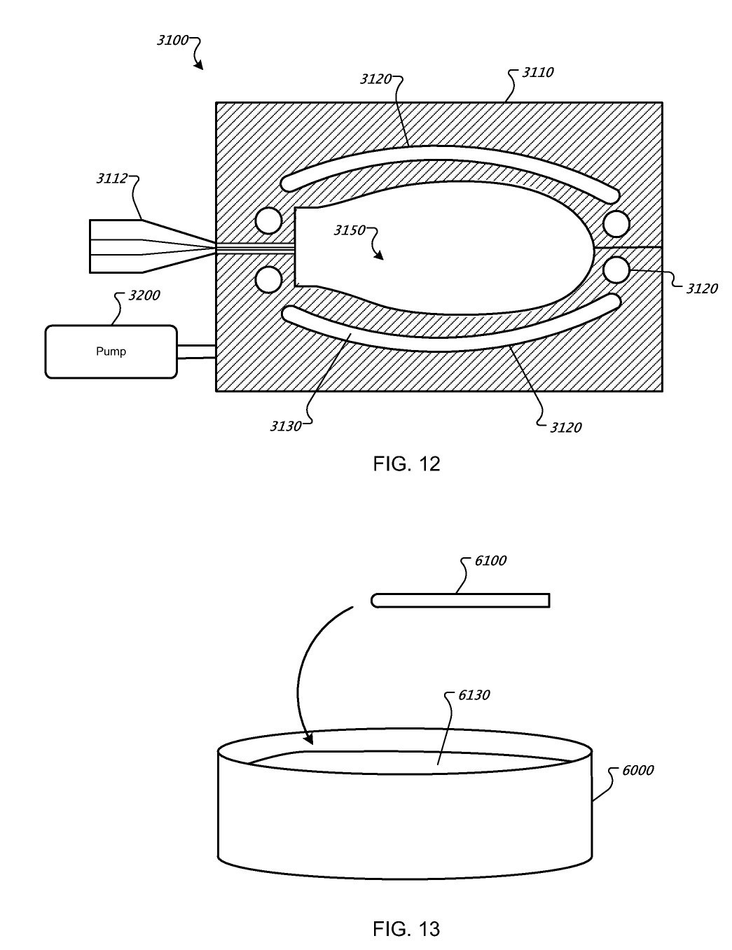

Referring to FIG. 12, in some implementations, a blow molding device 3100 (e.g. for a catheter) includes a blow mold manifold 3110 in fluid communication with at least one nozzle 3112. The nozzle 3112 is in fluid communication with a blow mold manifold 3110, which defines a blow mold cavity 3150 and at least one thermal conduction channel 3120. A pump 3200 is in fluid communication with the channel 3120 and pumps a liquid metal 3130 through the channel 3120 to control a blow molding device temperature. In some examples, the liquid metal 3130 comprises an alloy of gallium and indium, e.g. galistan. The ability of the liquid metal, e.g. galistan, to remain liquid at very low temperatures (e.g. about −20° C.) allows it to be used as a coolant for rapid cooling of the blow molding device 3100. The blow molding device 3100 may be an extension, injection, or stretch blow molding device. A molten polymer is injected through nozzle(s) 3112 into the heated preform mold cavity 3150 of the manifold 3110.

An extruded product is generally cooled after extrusion, which is often achieved by pulling the extrudate through a water bath. Plastics are very good thermal insulators and are therefore difficult to cool quickly. Referring to FIG. 13, in some implementations, a method of cooling an extruded polymer 6100 (e.g. a catheter) includes placing or pulling the extruded polymer 6100 into a bath 6000 of liquid metal, e.g. an alloy of gallium and indium, such as galistan, 6130 having a desired cooling temperature. Similarly, a method of heating an extruded polymer 6100 includes placing or pulling the extruded polymer 6100 into a bath 6000 of liquid metal, e.g. an alloy of gallium and indium, such as galistan, 6130 having a desired heating temperature. The method may be used for a common post-extrusion process called thermoforming, where the extrudate 6100 is heated until soft, and formed around a mold into a new shape.

A number of implementations have been described. Nevertheless, it will be understood that various modifications may be made without departing from the spirit and scope of the disclosure. Accordingly, other implementations are within the scope of the following claims.

What is claimed is:

1. A catheter comprising:
a longitudinally extending body having proximal and distal ends and defining at least one lumen, the lumen extending longitudinally from the proximal end through the body to the distal end and looping back to the proximal end; and
a liquid metal disposed in the lumen.

2. The catheter of claim 1, wherein the liquid metal comprises an alloy of gallium and indium.

3. The catheter of claim 2, wherein the liquid metal comprises galistan.

4. The catheter of claim 1 further comprising a power source in electrical communication with the liquid metal.

5. The catheter of claim 1, wherein the liquid metal is flowed though the lumen, a flow rate of the liquid metal controlling a catheter temperature.

6. The catheter of claim 1, wherein the lumen has a relatively narrower defined cross-section in a distal portion of the body than in a proximal portion of the body.

7. The catheter of claim 6, wherein the liquid metal is flowed though the lumen, the relatively narrower lumen in the distal portion of the body creating a flow resistance for the liquid metal.

8. The catheter of claim 6 further comprising a power source in electrical communication with the liquid metal, wherein the liquid metal provides an electrical conduit for current, the relatively narrower lumen in the distal portion of the body creating a current resistance for the liquid metal electrical conduit.

9. The catheter of claim 1, wherein a wall thickness between the lumen and an exterior surface of the body is relatively thinner in a distal portion of the body than in a proximal portion of the body, thereby allowing greater thermal conduction between the exterior surface of the body and the liquid metal about the distal portion of the body than about the proximal portion of the body.

10. The catheter of claim 1 further comprising a balloon disposed at the distal end of the body, a wall of the balloon defining a fluid channel in fluid communication with the lumen.

11. The catheter of claim 10, wherein the balloon fluid channel is in serial fluid communication with the lumen.

12. The catheter of claim 10, wherein the balloon fluid channel has a relatively narrower defined cross-section than the lumen.

13. The catheter of claim 10 further comprising a power source in electrical communication with the liquid metal, wherein the liquid metal provides an electrical conduit for current to heat tissue substantially about the balloon.

14. A catheter comprising:
a longitudinally extending body having proximal and distal ends and defining first and second lumens extending longitudinally through the body;
an electrically driven device coupled to the distal end of the body and in electrical communication with the first and second lumens;
a power source in electrical communication with the first and second lumens; and
a liquid metal disposed in the first and second lumens, the liquid metal providing an electrical conduit between the power source and electrically driven device.

15. The catheter of claim 14, wherein the liquid metal comprises an alloy of gallium and indium.

16. The catheter of claim 15, wherein the liquid metal comprises galistan.

17. The catheter of claim 14, wherein each lumen extends longitudinally from the proximal end through the body to the distal end and loops back to the proximal end, the liquid metal is flowed through the first and second lumens, thereby moving conducted heat away from a source of thermal conduction.

18. The catheter of claim 14 further comprising electrically insulative, thermally conductive particles disposed in the first and second lumens, the particles expanding upon heating and obstructing the first and second lumens to disjoin the liquid metal, severing the electrical conduit between the power source and electrically driven device.

19. The catheter of claim 18, wherein the particles expand to reversibly sever the electrical conduit.

20. The catheter of claim 19, wherein the particles comprise paraffin.

21. The catheter of claim 18, wherein the particles expand to irreversibly sever the electrical conduit.

22. The catheter of claim 21, wherein the particles comprise polymer microcapsules and a blowing agent.

23. A catheter comprising:
a longitudinally extending body having proximal and distal ends and defining first and second lumens, each lumen extending longitudinally from the proximal end through the body to the distal end and looping back to the proximal end;
an electrically driven device coupled to the distal end of the body and in electrical communication with the first and second lumens;
a power source in electrical communication with the first and second lumens; and
a liquid metal flowed though the first and second lumens, the liquid metal providing an electrical conduit between the power source and electrically driven device, wherein a flow rate of the liquid metal controls a catheter temperature.

24. The catheter of claim 23, wherein the liquid metal comprises an alloy of gallium and indium.

25. The catheter of claim 24, wherein the liquid metal comprises galistan.

26. An extruder head for an extruding device, comprising:
a head body defining at least one thermal conduction channel;
a pump in fluid communication with the channel; and
a liquid metal pumped through the channel to control an extruder head temperature.

27. The extruder head of claim 26, wherein the liquid metal comprises an alloy of gallium and indium.

28. The extruder head of claim 27, wherein the liquid metal comprises galistan.

29. A blow molding device comprising:
a manifold;
at least one nozzle in fluid communication with the manifold;
a blow mold in fluid communication with the nozzle, the blow mold defining a blow mold cavity and at least one thermal conduction channel;
a pump in fluid communication with the channel; and
a liquid metal pumped through the channel to control a blow molding device temperature.

30. The blow molding device of claim 29, wherein the liquid metal comprises an alloy of gallium and indium.

31. The blow molding device of claim 30, wherein the liquid metal comprises galistan.

32. A method of cooling an extruded polymer comprising placing the extruded polymer into a bath of liquid metal having a desired cooling temperature.

33. The method of claim 32, further comprising placing the extruded polymer in a bath of liquid metal comprising an alloy of gallium and indium.

34. The method of claim 33, wherein the liquid metal comprises galistan.

35. A method of heating an extruded polymer comprising placing the extruded polymer into a bath of liquid metal having a desired heating temperature.

36. The method of claim 35, further comprising placing the extruded polymer in a bath of liquid metal comprising an alloy of gallium and indium.

37. The method of claim 35, wherein the liquid metal comprises galistan.
